(19) United States
(12) Patent Application Publication
(10) Pub. No.: US 2010/0086515 A1
(43) Pub. Date: Apr. 8, 2010

(54) HUMAN IL-4 MUTEINS IN CANCER THERAPY

(75) Inventors: Thomas Höger, Laudenbach (DE); Jürgen Gamer, Dossenheim (DE)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: APOGENIX GMBH, Heidelberg (DE)

(21) Appl. No.: 12/306,518

(22) PCT Filed: Jul. 6, 2007
(86) PCT No.: PCT/EP2007/006026

§ 371 Date: Oct. 9, 2009

(30) Foreign Application Priority Data
Jul. 6, 2006 (EP) 06014080.3
Dec. 21, 2006 (EP) 06026609.5

(51) Int. Cl. A61K 38/20; A61P 35/00

(52) U.S. Cl. 424/85.2

(57) ABSTRACT

The present invention relates to the use of a combination of human interleukin-4 muteins and chemotherapeutic or pro-apoptotic agents for the prevention and/or treatment of cancer disease.

The present invention relates to the use of a combination of human interleukin-4 muteins and chemotherapeutic or pro-apoptotic agents for the prevention and/or treatment of cancer disease.

WO 2004/069274 refers to the use of cytokine antagonists which modulate the expression and/or the function of a cytokine in a cell for the downregulation of anti-apoptotic proteins in a cell. In particular, it is referred to the use of cytokine antagonists for the treatment of cancer. Muteins of the cytokines themselves are given as examples of cytokine antagonists, which are able to bind to the respective cell surface receptor, inhibiting the signal cascade triggered by the cytokine itself.

US Patents U.S. Pat. No. 6,313,272 and U.S. Pat. No. 6,028,176 describe recombinant agonistic or antagonistic human IL-4 muteins comprising at least one amino acid substitution in the binding surface of either the region of the A- or C-α-helix of the wild-type IL-4. The IL-4 muteins are indicated as being suitable for the treatment of condition exacerbated by IL-4 production such as asthma, allergy or inflammatory response-related conditions. It is speculated that the IL-4 muteins might be suitable for the treatment of cancers or tumours.

US Patent Application US 2005/0059590 describes modified IL-4 mutein receptor antagonists comprising an IL-4 mutein receptor antagonist, and in particular the IL-4 muteins as disclosed in the above-mentioned US Patents U.S. Pat. No. 6,313,272 and U.S. Pat. No. 6,028,176, coupled to polyethylene glycol. Said modified muteins are in particular indicated as useful in the treatment of severe asthma, chronic obstructive pulmonary disease and related lung conditions.

US Patent U.S. Pat. No. 5,723,118 describes mutant IL-4 proteins which compete with the wild-type IL-4 for occupation of the IL-4 receptor and act as antagonists or partial agonists of the human interleukin-4. In particular, mutant IL-4 proteins are disclosed wherein one or more of the amino acids occurring at position 121, 124 or 125 have been replaced. The mutant IL-4 proteins are indicated as being suitable for the treatment and/or prevention of allergic conditions.

US Patent U.S. Pat. No. 6,130,318 describes novel IL-4 antagonist or partial agonist mutant proteins and their use as medicaments, in particular in association with overshooting, falsely regulated immuno reactions and autoimmune diseases. Further, it is speculated that the IL-4 mutant proteins can be employed in the palliative therapy of tumour diseases.

It has now been found that antagonistic IL-4 muteins particularly as disclosed in the US Patents U.S. Pat. No. 6,313,272, U.S. Pat. No. 6,028,176, U.S. Pat. No. 5,723,118 and U.S. Pat. No. 6,130,318 and in the US Patent Application US 2005/0059590 are especially suitable in combination with at least one further chemotherapeutic or pro-apoptotic agent in the treatment of cancer diseases. Preferably, the antagonistic IL-4 muteins are used for curative cancer therapy.

Thus, the present invention refers to the use of a combination of (i) at least one human interleukin-4 mutein (IL-4 mutein) and (ii) at least one chemotherapeutic or pro-apoptotic agent for the manufacture of a medicament for the prevention and/or treatment of cancer.

The amino acid sequence of the IL-4 muteins differs from the amino acid sequence of the wild-type IL-4 by mutation of one or more single amino acids at certain positions of the native protein.

The term “mutation” as used in the context of the present invention can be understood as substitution, deletion and/or addition of single amino acids in the target sequence. Preferably, the mutation of the target sequence, in particular of the native IL-4, is a substitution at one or more positions of the native IL-4 polypeptide chain.

The substitution can occur with different genetically encoded amino acids or by non-genetically encoded amino acids. Examples for non-genetically encoded amino acids are homocysteine, hydroxyproline, ornithine, hydroxylysine, citrulline, carnitine, etc.

Moreover, in the context of this invention, a substitution within the native polypeptide sequence can be a conservative or a non-conservative substitution. The common classification of the amino acid residues on the base of the side-chain characteristics, which determine the amino acid groups for a conservative or a non-conservative substitution, is well known by the person skilled in the art.

In accordance with this invention, mutations of the native IL-4 amino acid sequence are being contemplated which result in IL-4 muteins having an antagonistic action with respect to the action of the wild-type IL-4. The term “antagonistic action” as used herein means that the IL-4 muteins of the invention are capable of modulating the function of the cytokine, in particular are capable of inhibiting the function of endogenous IL-4 cytokine. Autocrinely produced IL-4 in cancer cells promote the up-regulation of anti-apoptotic proteins which lead to resistance to cell death and to therapy refractoriness. Hence, an antagonistic action of the IL-4 muteins of the invention leads to the inhibition of the internal signal cascade triggered by the endogenous IL-4 which leads to the up-regulation of anti-apoptotic proteins.

Moreover, the IL-4 muteins of the invention may further show a higher affinity for the wild-type IL-4 receptor in comparison to wild-type IL-4. In particular, the muteins may compete with the endogenously expressed IL-4 for the binding site on the respective receptor.

Preferably, the present invention comprises the use of IL-4 muteins, wherein mutations of the amino acid sequence of the wild-type IL-4 sequence have been made to the region of the A-, C- and/or D-helices and more preferably to those amino acids comprising the binding surfaces of said helices of the IL-4 protein.

According to one preferred embodiment, the IL-4 mutein of the invention is preferably an IL-4 mutein as described in U.S. Pat. No. 5,723,118 and U.S. Pat. No. 6,130,318, which are herein incorporated by reference in their entirety.

Thus, a mutation to the region of the D-helix of the wild-type IL-4 protein sequence occurs preferably on at least one of the positions 120, 121, 122, 123, 124, 125, 126, 127 and/or 128 of the wild-type amino acid sequence. Even more preferably, the mutation occurs on at least one of the positions 121, 124 and/or 125. Most preferably, the mutation occurs at position 124.

In a very preferred embodiment of the present invention, a IL-4 mutein of the wild-type is used, wherein the amino acid tyrosine, which occurs naturally at position 124, is replaced by aspartic acid, glycine or glutamic acid, i.e. the Y124D-, the Y124G- and the Y124E-IL-4 mutein. Further, a IL-4 mutein is preferably used wherein the amino acid arginine, which occurs naturally at position 121, is replaced by aspartic acid or glutamic acid, i.e. the R121D- and R121E-IL-4 mutein. Further, a IL-4 mutein is preferably used wherein the amino acid serine, which occurs naturally at position 125, is replaced by aspartic acid or glutamic acid, i.e. the S125D- and S125E-IL-4 mutein.

According to a further preferred embodiment, the IL-4 mutein of the invention is an IL-4 mutein as described in U.S. Pat. No. 6,028,176 and U.S. Pat. No. 6,313,272, which are herein incorporated by reference in their entirety.

Thus, a mutation to the region of the A-helix of the wild-type IL-4 protein sequence occurs preferably on at least one of the positions 13 and 16. A mutation to the region of the C-helix of the wild-type IL-4 protein sequence occurs preferably on at least one of the positions 81 and 89.

In a very preferred embodiment of the present invention, a IL-4 mutein of the wild-type is used, wherein the amino acid threonine which occurs naturally at position 13 is replaced by aspartic acid, i.e. the T13D-IL-4 mutein. Further, a IL-4 mutein is preferably used wherein the amino acid serine which occurs naturally at position 16 is replaced by one of the amino acids selected from the group alanine, aspartate, isoleucine, leucine, glutane, arginine, threonine, valin, thyrosine (S16A-, S16D-, S16H-, S161-, S16L-, S16Q-, S16R-, S16T-, S16V- and S16Y-IL-4 mutein).

In a still further preferred embodiment, a IL-4 mutein is used, wherein the amino acid arginine which occurs naturally at position 81 is replaced by lysine, i.e. the R81K-IL-4 mutein. Still further, a IL-4 mutein is preferably used, wherein the amino acid aspargine, which occurs naturally at position 89, is replaced by isoleucine, i.e. the N89I-IL-4 mutein

Moreover, according to the present invention, IL-4 muteins are used which contain a combination of the above-disclosed mutations. According to a preferred embodiment of the invention, a IL-4 mutein is used which contains the mutation R121D and Y124D on the D-helix and in addition a third substitution on either the A- or C-helix. Preferably, the further mutations on either the A- or the C-helix are in nature and position as defined above.

Finally, the IL-4 muteins of the invention may be coupled to a non-protein polymer. In particular, as described in US 2005/0059590 and U.S. Pat. No. 6,130,312 (hereby incorporated by reference in their entirety), the IL-4 muteins may comprise additional amino acid substitutions, wherein said substitutions enable the site-specific coupling of at least one non-protein polymer. Examples for non-protein polymers are polyethylene glycol, polypropylene glycol or polyoxyalkylene.

In a preferred embodiment, the non-protein polymer is coupled to an amino acid residue and a residue at positions 28, 36, 37, 38, 104, 105 or 106 of the wild-type IL-4. In a still further embodiment, said positions in the wild-type IL-4 protein have been replaced by a cysteine.

It is further contemplated within the present invention to use as agent (i) IL-4 peptide mimetics that are capable to act as antagonists. For this purpose a peptide is designed which is capable of inhibiting the activity of IL-4 preventing the interaction of endogenous IL-4 with the specific IL-4 receptor. Suitable peptide mimetics are disclosed in US Patent U.S. Pat. No. 6,685,932 and US Patent Application US 2004/0030097, which are herein incorporated by reference in their entirety. In particular, said IL-4 peptide mimetics are designed in order to mime the helix A and helix C of the IL-4 cytokine, which are the residues involved in binding the specific IL-4 receptor.

The chemotherapeutic agents which are used in combination with the IL-4 mutein of the present invention preferably are antineoplastic compounds. Such compounds included in the present invention comprise, but are not restricted to, (a) antimetabolites, such as cytarabine, fludarabine, 5-fluoro-2′-deoxyuridine, gemcitabine, hydroxyurea or methotrexate; (b) DNA-fragmenting agents, such as bleomycin, (c) DNA-crosslinking agents, such as chlorambucil, platinum compounds, e.g. cisplatin, carboplatin or oxaliplatin, cyclophosphamide or nitrogen mustard; (d) intercalating agents such as adriamycin (doxorubicin) or mitoxantrone; (e) protein synthesis inhibitors, such as L-asparaginase, cycloheximide, puromycin or diphteria toxin; (f) topoisomerase I inhibitors, such as camptothecin or topotecan; (g) topoisomerase II inhibitors, such as etoposide (VP-16) or teniposide; (h) microtubule-directed agents, such as colcemide, colchicine, taxanes, e.g. paclitaxel, vinblastine or vincristine; (i) kinase inhibitors such as flavopiridol, staurosporine or derivatives thereof, e.g. STI571 (CPG 57148B) or UCN-01 (7-hydroxystaurosporine); (j) miscellaneous agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; (k) hormones such as glucocorticoids or fenretinide; (l) hormone antagonists, such as tamoxifen, finasteride or LHRH antagonists.

In an especially preferred embodiment of the present invention, the chemotherapeutic agent is selected from the group consisting of platinum compounds, e.g. cisplatin, doxorubicin and taxanes, e.g. paclitaxel.

The pro-apoptotic agents used in combination with IL-4 muteins of this invention are preferably TRAIL and CD95 ligands.

The IL-4 mutein in combination with the chemotherapeutic or pro-apoptotic agent may be administered locally or systematically. Preferably, the agents are administered parenterally, e.g. by injection or infusion, in particular intravenously, intramuscularly, transmucosally, subcutaneously or intraperitoneally. For this purpose, the IL-4 mutein is formulated as a pharmaceutical composition in a physiologically acceptable carrier, optionally together with physiologically acceptable excipients. The daily dose may vary depending on the mode of administration and/or the severity of the disease and is preferably in the range of 0.01 mg/kg to 100 mg/kg body weight. The combination therapy is carried out for a time period sufficient to obtain the desired beneficial effect, e.g. induction of a tumour response to treatment. The combined therapy should then be maintained until progression of the disease.

According to a preferred embodiment of the present invention, the administration of (i) at least one IL-4 mutein and (ii) at least one chemotherapeutic or pro-apoptotic agent may be simultaneous, separate or sequential, respectively. For example, the administration of agent (i) and agent (ii) is started simultaneously. Alternatively, the combination therapy can be started stepwise. According to one preferred embodiment of the present invention, the start of administration of the IL-4 mutein agent (i) is ≦1 week before the administration of the chemotherapeutic or pro-apoptotic agent (ii). The administration of the chemotherapeutic or pro-apoptotic agent (ii) may in turn start ≧1 week before the administration of the IL-4 mutein agent (i). The appropriate administration scheme of agent (i) and (ii) will be set up by a person skilled in the art, i.e. by a physician.

Moreover, the use of a combined therapy of the above agents (i) and (ii) which can further be in combination surgery and/or radiation therapy is also considered within the scope of this invention. In particular, the medicament combination is for simultaneous, separate or sequential combination therapy with surgery and/or radiation therapy.

Particularly, the IL-4 muteins in combination with the chemotherapeutic agent can be used for the treatment of cancer types classified as cytokine-expressing tumours and in particular cancer associated with increased IL-4 expression. Said cancer types may be at least partially resistant to apoptosis due to the expression of anti-apoptotic proteins. A method for the identification and diagnosis of cancer types and cells which express anti-apoptotic cysteines and which can be classified as cytokine-expressing tumours is disclosed in the European Patent Application EP 06 012 754. The teaching of said Application EP 06 012 754 is herein incorporated by reference in its entirety.

Examples of such cancer types comprise neuroblastoma, intestine carcinoma such as rectum carcinoma, colon carcinoma, familiary adenomatous polyposis carcinoma and hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, bladder carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeolid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidal melanoma, seminoma, rhabdomyosarcoma, craniopharyngeoma, osteosarcoma, chondrosarcoma, myosarcoma, liposarcoma, fibrosarcoma, Ewing sarcoma and plasmocytoma.

In a particularly preferred embodiment, the IL-4 mutein combination therapy according to the present invention can be used for the prevention and/or treatment of non-lymphoid and non-myeloid cancers, most preferably solid cancers, even more preferably epithelial cancers.

Especially preferred examples of epithelial cancer types include all forms of thyroid carcinomas (medullary thyroid carcinoma, papillary thyroid carcinoma, follicular thyroid carcinoma, anaplastic thyroid carcinoma), breast carcinoma, lung carcinoma, prostate carcinoma, bladder carcinoma, gastric carcinoma, pancreas carcinoma, kidney carcinoma, liver carcinoma and colon carcinoma.

In a further particularly preferred embodiment, the IL-4 mutein combination therapy according to the present invention is particularly useful for the prevention and/or treatment of minimal residual cancer disease (MRD). In fact, after cancer therapy, residual cancer cells often remain in the patient's body. These cancer cells can give rise to secondary cancers after the primary cancer has been removed. Therefore, one major task of successful cancer therapy must be the eradication of such residual cancer cells and in particular the eradication of cancer stem cells, e.g. colon cancer stem cells. In this context, the combination therapy of the present invention is therefore particularly suitable to reduce and/or eliminate residual cancer cells, in particular residual cancer stem cells, after an apparently complete regression or surgical excision of the primary tumour.

Further, the invention is explained in more detail by the following Example.

### EXAMPLE 1

### Effect of an IL-4 Mutein on the Growth of Colon and Breast Cancer Cells

The IL-4 double mutein R121 D/Y124D was tested on its effect on the growth of human colon and breast cancer cells in a mouse model.

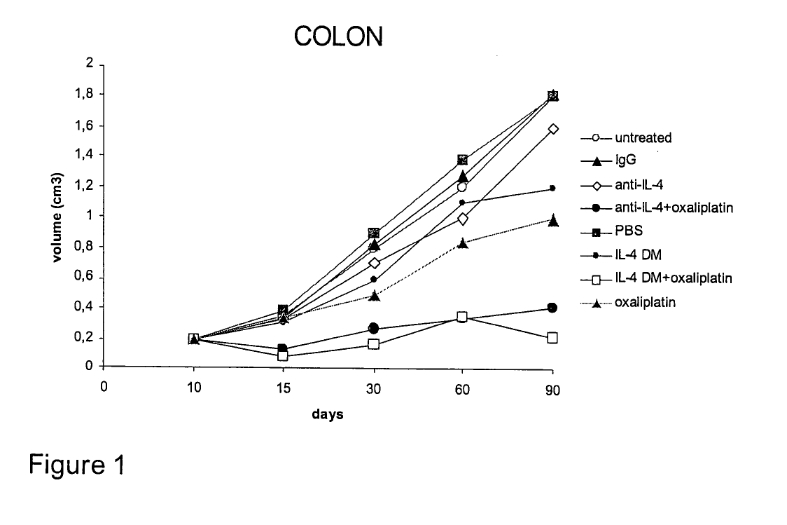

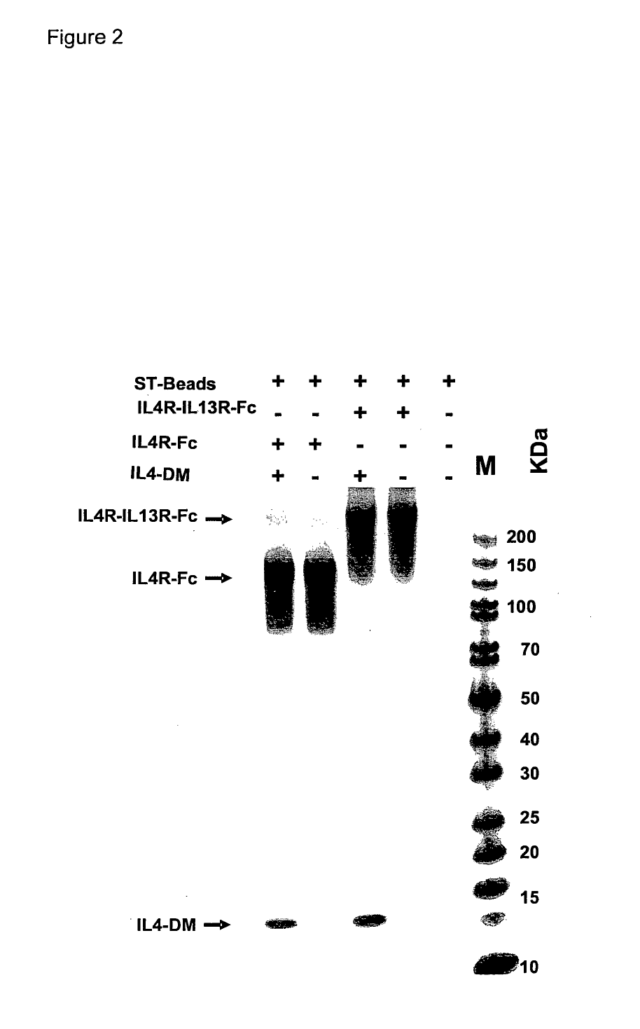

Human colon cancer stem cells (Ricci-Vitiani et al., Nature 2006, Nov. 19, Epub and ATCC CCL-248) and breast BT549 cancer cells (ATCC HTB-122) are positive for IL-4Rα and IL-4 expression at the protein as well as mRNA levels. These cells are primarily resistant to chemotherapy-cell death in vitro but they can be sensitized by anti-IL-4 treatment. Importantly, expression of IL-4 was maintained in subcutaneously grown tumours derived from human colon cancer stem cells and BT549 breast cancer cell line. We therefore treated nude mice (5 weeks old, female) carrying either human colon cancer stem cells or BT549 breast cancer line xenografts with IL-4 neutralising antibody alone (10 μg/cm³ on day 1 and day 4 for 3 weeks) or in combination with oxaliplatin or with doxorubicin (oxaliplatin: 0.40 mg/kg on day 1 every week for 4 weeks; doxorubicin: 6 mg/kg from day 1 once weekly for 4 weeks) and with IL-4 double mutein alone (30 μg/mouse twice a day for 10 days per 3 cycles) or in combination with oxaliplatin or doxorubicin, respectively. All mice were ip injected. The results are shown in FIG. 1 (colon cancer stem cells) and FIG. 2 (breast cancer cells).

When mice bearing human colon cancer stem cells or BT549 tumours were treated with anti-IL-4 or IL-4 double mutein alone, tumour growth was not significantly diminished. Similarly, when mice were treated with oxaliplatin or doxorubicin alone tumours growth were only marginally affected. These data indicate that treatment with IL-4-neutralising antibodies, oxaliplatin, doxorubicin or IL-4 double mutein alone is not sufficient to effectively prevent subcutaneous growth of colon and breast cancer xenografts. However, when anti-IL-4 or IL-4 double mutein was combined with oxaliplatin or doxorubicin to treat human colon cancer stem-tumour-bearing mice or to treat BT549-tumour-bearing mice respectively, growth was drastically reduced.

### EXAMPLE 2

### Receptor Binding Characteristics of an IL-Mutein

1) Expression and Purification of IL-4-Binding Proteins, IL4R-Fc and IL4R-IL13R-Fc

Hek 293T cells grown in DMEM+GlutaMAX (GibCo) supplemented with 10% FBS, 100 units/ml Penicillin and 100 μg/ml Streptomycin were transiently transfected with plasmids encoding fusion proteins, IL4R-Fc (a fusion of a soluble human IL4 receptor domain, a human IgG1 Fc domain and a Strep-Tag domain) and IL4R-IL13R-Fc (a fusion of a soluble human IL4 receptor domain, a soluble human IL13 receptor domain, a human IgG1 Fc domain and a Strep-Tag domain), respectively. A detailed description of these fusion proteins is found in PCT/EP2007/005480, which is herein incorporated by reference. Cell culture supernatants containing recombinant proteins were harvested three days post transfection and clarified by centrifugation at 300 g followed by filltration through a 0.22 μm sterile filter. For affinity purification Streptactin Sepharose was packed to a column (gel bed 1 ml), equilibrated with 15 ml buffer W (100 mM Tris-HCl, 150 mM NaCl pH 8.0) and the respective cell culture supernatant was applied to the column with a flow rate of 4 ml/min. Subsequently, the column was washed with buffer W and bound IL4R-Fc or IL4R-IL13R-Fc was eluted stepwise by addition of 6×1 ml buffer E (100 mM Tris-HCl, 150 mM NaCl, 2.5 mM desthiobiotin pH 8.0). The protein amount of the eluate fractions was quantified and peak fractions were concentrated by ultrafiltration and further purified by size exclusion chromoatography (SEC).

SEC was performed on a Superdex 200 column using an Äkta chromatography system (GE-Healthcare). The column was equilibrated with phosphate buffered saline and the concentrated, streptactin purified IL4R-Fc or IL4R-IL13R-Fc, respectively, were loaded onto the SEC column at a flow rate of 0.5 ml/min. The elution profile monitored by absorbance at 280 nm showed a prominent protein peak at 10.31 ml for IL4R-IL13R-Fc and 12.97 ml for IL4R-Fc. SEC fractions for IL4R-Fc were additionally analysed under denaturing conditions by SDS-PAGE and silver staining.

2) IL4 Mutein Pull Down Assay

To test for specific binding of the IL-4-double mutein R121D/Y124D to IL4R-Fc and IL4R-IL13R-Fc, 4 μg of both Fc fusion proteins, respectively, were immobilized to Streptactin Sepharose (ST) via their Strep-Tag domain. The immobilized proteins were subsequently incubated for 60 min with 400 ng of purified human IL-4-double mutein R121D/Y124D (IL-4 DM) in a total volume of 400 μl phosphate buffered saline. Subsequently, the beads were washed and bound proteins were specifically eluted with desthiobiotin in a total volume of 40 μl elution buffer. Eluted proteins were finally analysed via SDS-PAGE and silver staining. As shown in FIG. 2 IL-4 DM shows specific binding to both IL-4 receptor constructs IL4R-Fc and IL4R-IL13R-Fc indicated by the presence of IL-4 DM protein (12 kDa) that could not be seen in control experiments.

1. Use of a combination comprising:
(i) a human interleukin (IL)-4 mutein, and
(ii) a chemotherapeutic or pro-apoptotic agent for the manufacturing of a medicament for the prevention or treatment of cancer.

2. The use of claim 1, wherein the IL-4 mutein comprises a mutation in the region of the A-, C- or D-helix of the wild-type IL-4 protein of SEQ ID NO:1.

3. The use of claim 2, wherein the IL-4 mutein comprises a mutation in the region of the D-helix in an amino acid residue selected from the group consisting of the amino acid residues at position 120, 121, 122, 123, 124, 125, 126, 127 and 128 of the wild-type IL-4 protein of SEQ ID NO:1.

4. The use of claim 3, wherein the mutation occurs at the amino acid residue at position 121, 124 or 125.

5. The use of claim 4, wherein the IL-4 mutein comprises the amino acid sequence of SEQ ID NO:2, SEQ ID NO:3, SEQ ID NO:4, or SEQ ID NO:5.

6. The use of claim 4, wherein the IL-4 mutein comprises the amino acid sequence of SEQ ID NO:6 or SEQ ID NO:7.

7. The use of claim 4, wherein the IL-4 mutein comprises the amino acid sequence of SEQ ID NO:8 or SEQ ID NO:9.

8. The use of claim 2, wherein the IL-4 mutein comprises a mutation in the region of the A-helix in an amino acid residue selected from the group consisting of the amino acid residues at position 13 and 16 of the wild-type IL-4 protein of SEQ ID NO:1.

9. The use of claim 8, wherein the IL-4 mutein comprises the amino acid sequence of SEQ ID NO:22.

10. The use of claim 8, wherein the IL-4 comprises the amino acid sequence of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, or SEQ ID NO:19.

11. The use of claim 2, wherein the IL-4 mutein comprises a mutation in the region of the C-helix in an amino acid residue selected from the group consisting of amino acid residues at position 81 and 89 of the wild-type IL-4 protein of SEQ ID NO:1.

12. The use of claim 11, wherein the IL-4 mutein comprises the amino acid sequence of SEQ ID NO:20.

13. The use of claim 11, wherein the IL-4 mutein comprises the amino acid sequence of SEQ ID NO:21.

14. The use of claim 2, wherein the IL-4 mutein comprises
(i) the mutations R121D and Y124D in the D-helix of the wild-type IL-4 protein of SEQ ID NO:1; and
(ii) at least one amino acid mutation on either the A- or C-helices of the wild-type IL-4 protein of SEQ ID NO:1.

15. The use of claim 14, wherein the mutation on either the A- or C-helices is selected from the group consisting of amino acid mutations at position 13, 16, 81 and 89 of the wild-type IL-4 protein of SEQ ID NO:1.

16. The use of claim 1, wherein the IL-4 mutein is coupled to a non-protein polymer.

17. The use of claim 16, wherein the non-protein polymer is coupled to the IL-4 mutein at an amino acid residue at position 28, 36, 37, 38, 104, 105 or 106 of an IL-4 mutein having an amino acids sequence of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, SEQ ID NO:18, SEQ ID NO:19, SEQ ID NO:20, SEQ ID NO:21, or SEQ ID NO:22, and wherein the non-protein polymer is polyethylene glycol, polypropylene glycol or a polyoxyalkylene.

18. The use of claim 17, wherein the amino acid residue at position 28, 36, 37, 38, 104, 105 or 106 is replaced by cysteine.

19. The use of claim 1, wherein the chemotherapeutic agent is selected from the group consisting of an antimetabolite, a DNA-fragmenting agent, a DNA-crosslinking agent, an intercalating agent, a protein synthesis inhibitor, a topoisomerase inhibitor I, a topoisomerase inhibitor II, a microtubule-directed agent, a kinase inhibitor, a hormone and a hormone antagonist.

20. The use of claim 19, wherein the chemotherapeutic agent is selected from the group consisting of a taxane, a platinum compound and doxorubicin.

21. The use of claim 1, wherein the pro-apoptotic agent is selected from the group consisting of a TRAIL ligand and a CD95 ligand.

22. The use of claim 1, wherein administration of the human IL-4 mutein and the chemotherapeutic or proapoptotic agent to a cancer patient is simultaneous, separate or sequential.

23. The use of claim 22, wherein the medicament is for simultaneous, separate or sequential combination therapy with surgery or radiation therapy.

24. The use of claim 1, wherein the medicament further comprises a pharmaceutical acceptable carrier or excipient.

25. The use of claim 22, wherein the cancer patient has a cancer which has been classified as a cytokine-expressing cancer.

26. The use of claim 25, wherein the cancer is a solid tumor or an epithelial cancer.

27. The use of claim 26, wherein the cancer is selected from the group consisting of thyroid carcinoma, breast carcinoma, lung carcinoma, prostate carcinoma, bladder carcinoma, gastric carcinoma, pancreas carcinoma, kidney carcinoma, liver carcinoma and colon carcinoma.

28. The use of claim 27, wherein the thyroid carcinoma is selected from the group consisting of a medullary thyroid carcinoma, a papillary thyroid carcinoma, a follicular thyroid carcinoma and an anaplastic thyroid carcinoma.

29. The use of claim 22, wherein the cancer is a minimal residual cancer.

30. A method for the treatment of a non-lymphoid or non-myeloid cancer expressing interleukin (IL)-4, the method comprising the step of:
(a) selecting a subject having a non-lymphoid or non-myeloid cancer producing IL-4;
(b) contacting the non-lymphoid or non-myeloid cancer with an IL-4 mutein comprising an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, SEQ ID NO:18, SEQ ID NO:19, SEQ ID NO:20, SEQ ID NO:21 and SEQ ID NO:22; and
(c) contacting the non-lymphoid or non-myeloid cancer with a chemotherapeutic or pro-apoptotic agent.

31. The method according to claim 30, wherein the IL-4 mutein is coupled to a non-protein polymer.

32. The method according to claim 31, wherein the non-protein polymer is selected from the group consisting of polyethylene glycol, polypropylene glycol and polyoxyalkylene.

33. The method according to claim 31, wherein the non-protein polymer is coupled to the IL-4 mutein at an amino acid residue at position 28, 36, 37, 38, 104, 105 or 106.

34. The method according to claim 33, wherein the amino acid residue at position 28, 36, 37, 38, 104, 105 or 106 is replaced by cysteine.

35. The method according to claim 30, wherein the chemotherapeutic s agent is selected from the group consisting of an antimetabolite, a DNA-fragmenting agent, a DNA-crosslinking agent, an intercalating agent, a protein synthesis inhibitor, a topoisomerase inhibitor I, a topoisomerase inhibitor II, a microtubule-directed agent, a kinase inhibitor, a hormone and a hormone antagonist.

36. The method according to claim 30, wherein the chemotherapeutic agent is selected from the group consisting of a taxane, a platinum compound and doxorubicin.

37. The method according to claim 36, wherein the platinum compound is oxaliplatin.

38. The method according to claim 30, wherein the pro-apoptotic agent is selected from the group consisting of a TRAIL ligand and a CD95 ligand.

39. The method according to claim 30, wherein step (b) and step (c) are performed simultaneously, separate or sequential.

40. The method according to claim 30, further comprising surgery or radiation therapy.

41. The method according to claim 30, wherein the non-lymphoid or non-myeloid cancer is an epithelial cancer.

42. The method according to claim 30, wherein the non-lymphoid or non-myeloid cancer is selected from the group consisting of neuroblastoma, intestine carcinoma, rectum carcinoma, colon carcinoma, familiary adenomatous polyposis carcinoma, hereditary non-polyposis colorectal cancer, esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, follicular thyroid carcinoma, anaplastic thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, bladder carcinoma, melanoma, brain tumor, glioblastoma, astrocytoma, meningioma, medulloblastoma, peripheral neuroectodermal tumor, Hodgkin lymphoma, non-Hodgkin lymphoma, Burkitt lymphoma, acute lymphatic leukemia (ALL), chronic lymphatic leukemia (CLL), acute myeolid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidal melanoma, seminoma, rhabdomyosarcoma, craniopharyngeoma, osteosarcoma, chondrosarcoma, myosarcoma, liposarcoma, fibrosarcoma, Ewing sarcoma and plasmocytoma.

43. The method according to claim 30, wherein the non-lymphoid or non-myeloid cancer is a minimal residual cancer.

44. The method according to claim 30, wherein the wherein the non-lymphoid or non-myeloid cancer is at least partially resistant to apoptosis.

45. The method according to claim 30, wherein the non-lymphoid or non-myeloid cancer autocrinely produces the IL-4.

46. A method for inhibiting the function of interleukin (IL)-4 in a subject, the method comprising the step of:
(a) selecting a subject having a non-lymphoid or non-myeloid cancer expressing IL-4; and
(b) contacting the non-lymphoid or non-myeloid cancer with an IL-4 mutein comprising an amino acid sequence selected from the group consisting of SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14 SEQ ID NO:15, SEQ ID NO:16, SEQ ID NO:17, SEQ ID NO:18, SEQ ID NO:19, SEQ ID NO:20, SEQ ID NO:21 and SEQ ID NO:22;
whereby the function of the interleukin in the subject is inhibited.


SEQUENCE LISTING (22 sequences)

SEQ ID NO: 1 - protein, 129 aa, Homo sapiens [sequence omitted]

SEQ ID NO: 2 - protein, 129 aa, Artificial Sequence
  R121D/Y124D-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 3 - protein, 129 aa, Artificial Sequence
  Y124D-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKDSKCSS

SEQ ID NO: 4 - protein, 129 aa, Artificial Sequence
  Y124G-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKGSKCSS

SEQ ID NO: 5 - protein, 129 aa, Artificial Sequence
  Y124E-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKESKCSS

SEQ ID NO: 6 - protein, 129 aa, Artificial Sequence
  R121D-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKYSKCSS

SEQ ID NO: 7 - protein, 129 aa, Artificial Sequence
  R121E-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMEEKYSKCSS

SEQ ID NO: 8 - protein, 129 aa, Artificial Sequence
  S125D-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYDKCSS

SEQ ID NO: 9 - protein, 129 aa, Artificial Sequence
  S125E-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYEKCSS

SEQ ID NO: 10 - protein, 129 aa, Artificial Sequence
  S16A-IL-4 mutein
HKCDITLQEIIKTLNALTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 11 - protein, 129 aa, Artificial Sequence
  S16D-IL-4 mutein
HKCDITLQEIIKTLNDLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 12 - protein, 129 aa, Artificial Sequence
  S16H-IL-4 mutein
HKCDITLQEIIKTLNHLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 13 - protein, 129 aa, Artificial Sequence
  S16I-IL-4 mutein
HKCDITLQEIIKTLNILTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 14 - protein, 129 aa, Artificial Sequence
  S16L-IL-4 mutein
HKCDITLQEIIKTLNLLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 15 - protein, 129 aa, Artificial Sequence
  S16Q-IL-4 mutein
HKCDITLQEIIKTLNQLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 16 - protein, 129 aa, Artificial Sequence
  S16R-IL-4 mutein
HKCDITLQEIIKTLNRLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 17 - protein, 129 aa, Artificial Sequence
  S16T-IL-4 mutein
HKCDITLQEIIKTLNTLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 18 - protein, 129 aa, Artificial Sequence
  S16V-IL-4 mutein
HKCDITLQEIIKTLNVLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 19 - protein, 129 aa, Artificial Sequence
  S16Y-IL-4 mutein
HKCDITLQEIIKTLNYLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 20 - protein, 129 aa, Artificial Sequence
  R81K-IL-4 mutein
HKCDITLQEIIKTLNSLTEQKTLCTELTVTDIFAASKNTTEKETFCRAATVLRQFYSHHEKDTRCLGATAQQFHRHKQLIKFLKRLDRNLWGLAGLNSCPVKEANQSTLENFLERLKTIMREKYSKCSS

SEQ ID NO: 21 - protein, 129 aa, Artificial Sequence
  N89I-IL-4 mutein
HKCDITLQEIIKTLNSLTEQKTLCTELTVTDIFAASKNTTEKETFCRAATVLRQFYSHHEKDTRCLGATAQQFHRHKQLIRFLKRLDRILWGLAGLNSCPVKEANQSTLENFLERLKTIMREKYSKCSS

SEQ ID NO: 22 - protein, 129 aa, Artificial Sequence
  T13D-IL-4 mutein
HKCDITLQEIIKDLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS